(19) United States
(12) Patent Application Publication
(10) Pub. No.: US 2003/0175151 A1
(43) Pub. Date: Sep. 18, 2003

(54) Venous blood reservoir in an extracorporeal circuit

(76) Inventors: Nicola Ghelli, S. Pietro in Casale (IT); Ivo Panzani, Mirandola (IT); Gabriele Tommasi, Cavezzo (IT)

Correspondence Address: POPOVICH & WILES, PA, 80 SOUTH 8TH STREET, SUITE 1902, MINNEAPOLIS, MN, 55402

(21) Appl. No.: 10/382,692

(22) Filed: Mar. 6, 2003

(30) Foreign Application Priority Data
Mar. 12, 2002 (IT) MI2002A000526

(51) Int. Cl. A61M 1/14; A61M 37/00

(52) U.S. Cl. 422/44; 604/6.15; 604/6.1; 604/4.01

(57) ABSTRACT

A vacuum assisted venous blood reservoir has a controller that can sense pressure within a sealed blood chamber. The controller regulates the amount of vacuum within the chamber to control the level of blood in the venous reservoir. A safety valve is included to prevent blood from entering the vacuum line.

### FIELD OF THE INVENTION

The present invention relates to a venous blood reservoir in extracorporeal circuit.

### BACKGROUND OF THE INVENTION

It is known that many surgical procedures entail the need to divert the blood of the patient into an extracorporeal circuit that comprises blood accumulation reservoirs. One of the reservoirs is the so-called venous reservoir, which receives the blood, known as venous blood, from the patient before it is passed through an oxygenation apparatus, which is also included in the extracorporeal circuit. Another of the reservoirs is designed to contain blood collected in the operating field, which once appropriately filtered can be returned to the patient; this reservoir is known as a cardiotomy reservoir.

The venous reservoirs of the prior art receive the blood that arrives from the patient by gravity, and this entails a forced placement of such reservoirs at a lower level than the operating field, which is often poorly compatible with the limited space available. It should also be noted that the blood circulation provided by gravity is not always as efficient as would be desirable. One way to increase the efficiency of drainage is to apply a vacuum to the interior of the reservoir. However, such vacuum assisted reservoirs have not, in the past, been combined with a cardiotomy reservoir to form a reservoir system having features desired by the user.

### SUMMARY OF THE INVENTION

The aim of the present invention is therefore to provide a venous reservoir that ensures intensive drainage of the blood of the patient without any limitation in selecting its location. Within this aim, an object of the invention is to devise a venous reservoir combined with a cardiotomy reservoir.

The proposed aim is achieved by a venous blood reservoir according to the invention, comprising an inlet connector that is connected to a line for drawing blood from the patient, and an outlet connector for the outflow of the blood toward an oxygenation apparatus, characterized in that it comprises, at the upper lid, a connector that is adapted to be connected to a line that reaches a vacuum source.

### BRIEF DESCRIPTION OF THE DRAWINGS

Further characteristics and advantages will become better apparent from the description of a preferred but not exclusive embodiment of the invention, illustrated only by way of non-limitative example in the accompanying drawings, wherein:

FIG. 1 is a general sectional view of the device of this invention, taken along the line I-I of FIG. 2;

FIG. 2 is a sectional view, taken along the line II-II of FIG. 1;

FIG. 3 is an enlarged-scale view of the upper region of FIG. 2 in a different functional condition; and

FIG. 4 is a detail view of the device of this invention.

### DETAILED DESCRIPTION OF THE PREFERRED EMBODIMENTS

With reference to the figures, reference number 1 designates the enclosure of the combined device. Enclosure 1 includes a first chamber or portion comprising a venous reservoir 2 and a second chamber or portion comprising a cardiotomy reservoir 4. Venous reservoir 2 is positioned below cardiotomy reservoir 4 and the two reservoirs are separated by a generally horizontal partition 3. It should be understood, however, that the concepts of the present invention disclosed herein are not limited to a venous reservoir combined with a cardiotomy reservoir but are equally applicable to stand-alone venous reservoirs.

The venous reservoir 2 is provided with an inlet connector 5 for the inflow of the venous blood, into a central region 5a that is defined by a wall 6. Wall 6 comprises a bubble rupturing substance and is surrounded by a filter 6a. Venous reservoir 2 is also provided with an outlet connector 7 for the outflow of the blood. Thus, blood entering venous reservoir 2 through inlet connector 5 is directed to central region 5a. Before the blood exits the venous reservoir, it passes through wall 6 and filter 6a where bubbles and other undesirable materials are removed.

The cardiotomy reservoir 4 is provided with an inlet connector 8 for the inflow of the blood that arrives from the operating field. Inlet connector 8 opens into a region 8a that is defined by wall 9, which comprises a bubble rupturing substance. Wall 9 is surrounded by a filter 9a.

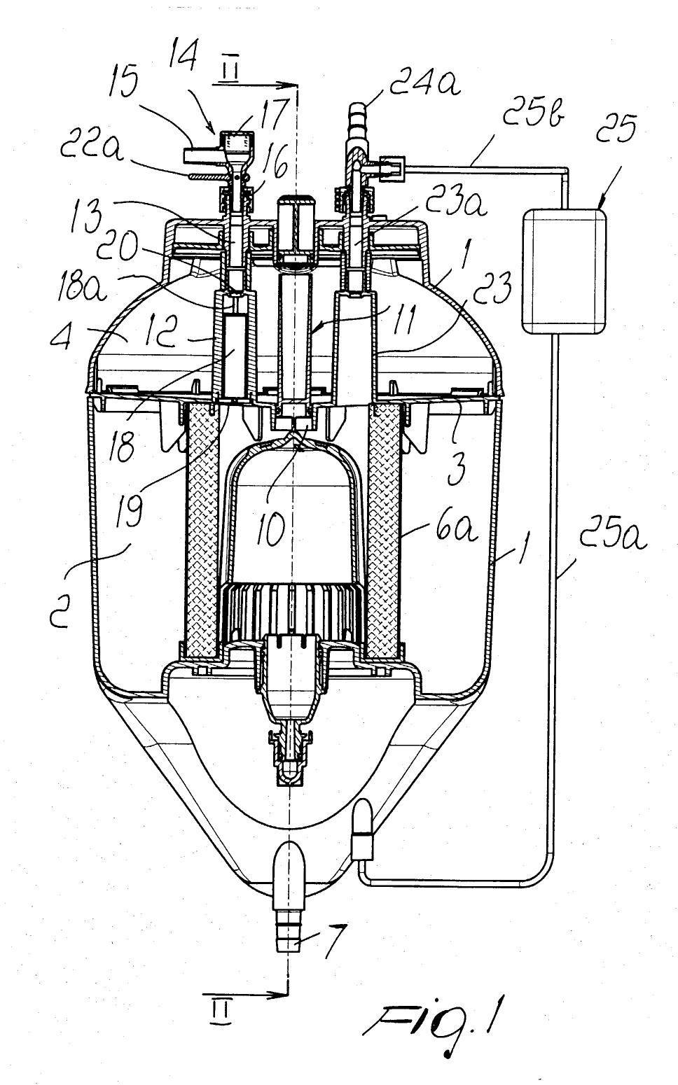

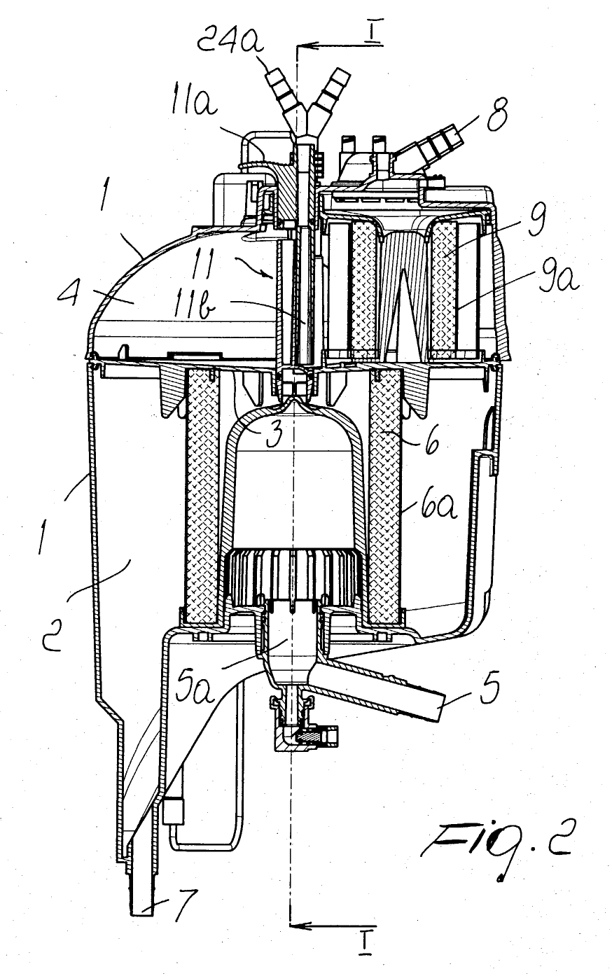

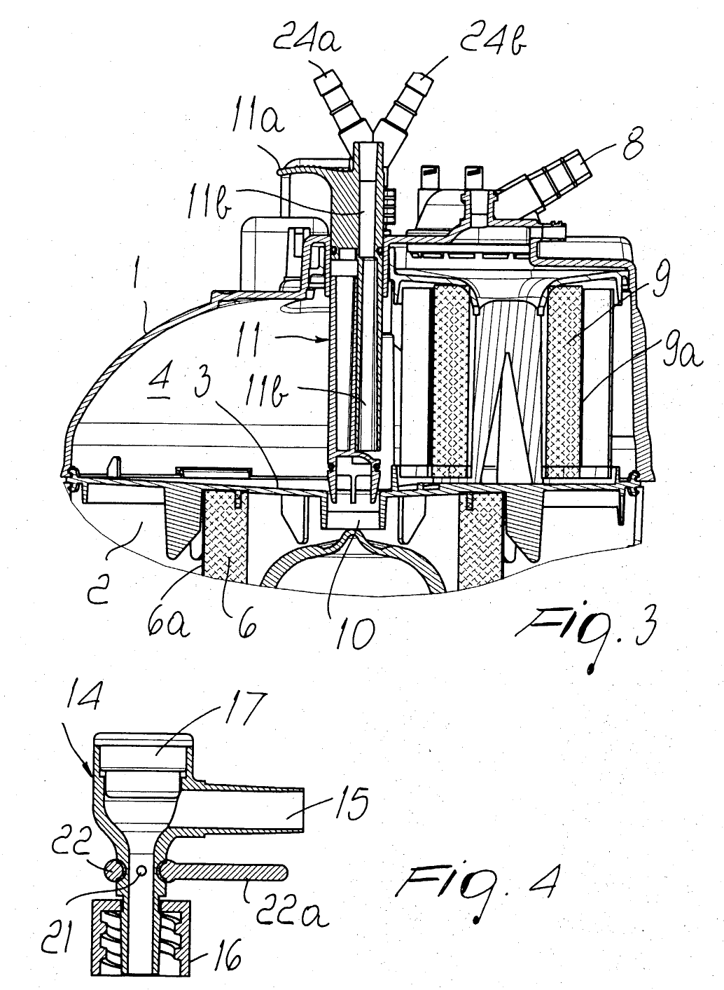

The partition 3 has a central opening 10, which connects the cardiotomy reservoir 4 to the venous reservoir 2. Means are provided for closing central opening 10. Manual action on tab 11 a will cause column 11 to slide between a lower stroke limit position (as shown in FIGS. 1 and 2), thus closing opening 10, and an upper stroke limit position ( as shown in FIG. 3), in which opening 10 is open in order to allow emptying of the blood contained in the cardiotomy reservoir into the underlying venous reservoir.

If it is necessary to empty the cardiotomy reservoir while opening 10 is closed, the duct 11b provided in the column 11 is used. Duct 11b is open at the lower end into the cardiotomy reservoir to allow aspiration through the open upper end.

An important feature of the invention is that partition 3, comprising the upper lid of the venous reservoir 2, is provided with a connector 12, which comprises a duct which communicates with a lower end of duct 13 formed within the upper structure of the enclosure 1. As best seen in FIG. 4, an upper end of duct 13 joins coupling 14 which has a vacuum port comprising a connector 15 that is adapted to be connected to a line that is attached to a vacuum source.

The connection of the coupling 14 to the upper end of the duct 13 is provided by means of the threaded ring 16, which allows rotation of the coupling. A safety valve 17 contained within coupling 14 prevents vacuum from exceeding a desired level.

During use of the device, vacuum is applied to venous reservoir 2 by connecting connector 15 to a vacuum source. The formation of vacuum in the venous reservoir 2 results in an effective drainage of the blood that arrives from the patient, and this occurs regardless of the level at which the venous reservoir is arranged with respect to the operating field.

As best seen in FIG. 1, within the connector 12 there is a float 18, which in the inactive or open position (shown in FIG. 1) rests on a frame 19. Float 18 is provided with needle 18a, which is adapted to close an opening 20 that connects the connector 12 to the duct 13 when float 18 is moved to the active or open position. If the blood contained in the venous reservoir 2 reaches a level where it enters connector 12 creating a situation that might lead to invasion of the vacuum line on the part of the blood, float 18 will be caused by the blood to move upwardly in turn moving needle 18a to a position which blocks opening 20 to duct 13, thus preventing blood from entering the vacuum line.

The return of the float 18 to the inactive position, once the emergency has ended, is facilitated by the temporary connection of the space within duct 13 that lies above the float to atmospheric pressure. This is accomplished by opening a small hole 21 provided in the coupling 14 by deformation of an elastic ring 22, which normally keeps the hole closed, the deformation being caused by the operator by manually acting on a tab 22a.

A turret 23 opens into venous reservoir 2 and extends from partition 3. It is joined by means of duct 23a to connectors 24a and 24b for connection to lines that are normally closed in order to maintain a desired amount of vacuum in the reservoir 2. The turret is adapted for the introduction, when necessary, of medical liquids into the reservoir.

The venous reservoir according to the invention is provided with a control system comprising a device capable of automatic adjustment of the level of the blood contained therein based upon pressure within the reservoir. The control system includes a computer schematically designated by the reference numeral 25. The pressure at the base of the reservoir and the negative pressure at the top of the reservoir are continuously sensed and provided to the computer. The pressure at the top and bottom of the reservoir is sensed through ducts 25a and 25b, respectively. The computer adjusts the amount or degree of vacuum that is present in the reservoir based on the sensed pressure data to maintain the proportion between the flow-rate of the incoming blood and the flow-rate of the outgoing blood, to keep the level of the blood in the venous reservoir substantially constant.

The described invention is susceptible of numerous modifications and variations, all of which are within the scope of the inventive concept; thus, for example, the device for automatic adjustment of the level of the blood in the reservoir can be provided in any manner and the cardiotomy reservoir can be omitted.

Furthermore, connector 15 adapted to be connected to the vacuum line can branch out from the wall of the enclosure 1 proximate to the partition 3.

What is claimed is:

1. A vacuum assisted venous blood reservoir comprising:
a sealed blood chamber having a blood inlet and a blood outlet;
a vacuum port connected at an upper portion of the chamber, the vacuum port adapted for connection to a vacuum source; and
a valve having an open position which allows fluid flow between the vacuum port and the chamber and a closed position which blocks fluid flow between the vacuum port and the chamber, the valve moving from the open position to the closed position without operator intervention when a level of blood in the chamber rises above a predetermined level.

2. The vacuum assisted venous blood reservoir of claim 1 wherein the valve is a float valve.

3. The vacuum assisted venous blood reservoir of claim 2 wherein the float valve comprises a needle adapted to move between the open position and the closed position.

4. The vacuum assisted venous blood reservoir of claim 1 further comprising a device for automatic adjustment of the level of the blood contained in the chamber.

5. The vacuum assisted venous blood reservoir of claim 4 wherein the device for automatic adjustment of the level of the blood continuously senses the pressure in the chamber.

6. A vacuum assisted venous reservoir system adapted to be connected to a vacuum source, the vacuum assisted venous reservoir system comprising:
a sealed blood chamber having a blood inlet and a blood outlet;
a vacuum port connected at an upper portion of the chamber, the vacuum port adapted for connection to the vacuum source; and
a vacuum controller connected to sense pressure in at least two locations within the blood chamber and, in response to the sensed pressure, to regulate the amount of vacuum applied by the vacuum source at the vacuum port.

7. The system of claim 6 wherein the two locations are an upper portion of the blood chamber and a lower portion of the blood chamber.
